(19) United States
(12) Patent Application Publication
(10) Pub. No.: US 2004/0198695 A1
(43) Pub. Date: Oct. 7, 2004

(54) Joint-protecting beverages and/or foods and their preparations

(76) Inventors: Anhu Li, Shanghai (CN); Qingren Xu, Shanghai (CN)

Correspondence Address: RABIN & BERDO, P.C., 1101 14 Street, N.W., Suite 500, Washington, DC, 20005, US

(21) Appl. No.: 10/793,035

(22) Filed: Mar. 5, 2004

(30) Foreign Application Priority Data
Apr. 2, 2003 (CN) 03116133.2

(51) Int. Cl. A61K 31/737; A61K 31/7008; A61K 31/522; A61K 31/355

(52) U.S. Cl. 514/54; 514/167; 514/62; 514/263.32; 514/557; 514/474; 514/458; 514/725

(57) ABSTRACT

A series of beverages and/or foods, which have protecting and enhancing effects on human's joints and bones and can therefore prevent and treat osteoarthritis, is provided. The beverages and/or foods comprise one or more cartilage- and synovial fluid-enhancing active ingredients or combinations of these ingredients with bone-enhancing calcium and phosphorus additives. Active ingredients in these beverages and/or foods are taken in liquid forms. So they can be easily absorbed and utilized thoroughly by human body.

### FIELD OF THE INVENTION

The present invention falls into the food supplements area. More specifically, this invention relates to a series of joint-protecting beverages and/or foods and their preparations.

### BACKGROUND

Arthritis is one of human's most difficult-to-treat diseases. Among the many variants of arthritis, osteoarthritis is by far the most common one. The causes of osteoarthritis include heritage, aging, repeated joint overuse, obesity, etc. Aging is the leading cause for this disease. With age increases, one's cartilage and synovial fluid of joints degenerate gradually and eventually become osteoarthritis. Besides age, joint damage caused by sports-related events, such as athletic training or heavy physical labor, is another major cause of osteoarthritis. Currently, more than 40 million Americans are affected by some form of arthritis and over 20 millions of them suffer from osteoarthritis. So, it is very important to develop an effective, safe, and simple way for treating and preventing arthritis.

In the past, NSAIDs (non-steroidal anti-inflammatory drugs), such as aspirin, ibuprofen, naproxen, nabumetone, etc, were traditional treatments for osteoarthritis. The anti-inflammatory activities of NSAIDs come from the inhibition of cyclooxygenases (COXs), which are rate-determining enzymes in the synthesis of prostaglandin. COXs exist in two isoforms, i.e., COX1 and COX2. The former is expressed almost everywhere in the body, but the expression level of the latter in most cells is very low except for in the inflammatory sites. Traditional NSAIDs, due to lack of selectivity between COXI and COX2, often cause severe stomach adverse effects, such as stomachache, ulcer, diarrhea, etc (Cryer, B.; Feldman, M. Am. J. Med. 1998, 104(5), 413-421). Acetaminophen later became widely used for treating arthritis because of its reduced adverse effects and similar effectiveness with NSAIDs. More recent treatments for arthritis are Celebrex and Vioxx, which are COX2-selective inhibitors and therefore have fewer side effects (Lee, S.; Simon, M. D. Osteoarthritis: A Review: Clinical Cornerstone 1999, 2(2), 23-24). However, all the above-mentioned drugs can only improve clinical symptoms of arthritis and cannot prevent or slow down degeneration of cartilage and synovial fluid in joints. Furthermore, these drugs can potentially cause stomach and cardiovascular problems.

Glucosamine, currently being used as arthritis treatments in Europe, America and some Asian countries, has proven to be an effective and safe drug. To date, more than 20 clinical trials that involved 6,000 patients have been performed worldwide to test the effectiveness and safety of glucosamine (some results see: Brief, A. A.; Maurer, S. G.; Di Cesare, P. E. J. Am. Acad. Orthop. Surg. 2001, 9, 71-78). Glucosamine exists naturally in human body. It is the raw material for synthesizing three major components of cartilage, i.e., glucosaminoglycans, proteoglycans and chondroitin sulfate. Glucosamine is also the basic building block for synthesizing hyaluronic acid, the main component of synovial fluid. Therefore, glucosamine is the necessary ingredient and basic nutrition for a healthy joint. With age increases, human body's ability to generate glucosamine decreases. Degeneration of cartilage and synovial fluid happens due to insufficient supplies of glucosamine and eventually leads to osteoarthritis. Supplementing glucosamine externally is an effective way to prevent and treat osteoarthritis through restoring damaged cartilage and synovial fluid. In addition to its repairing function, glucosamine has been clinically proven to be able to improve the inflammatory situation caused by athletic training or heavy physical labor. World Health Organization has officially classified glucosamine sulfate as a slow-acting drug for the treatment of osteoarthritis.

The following are other drugs that have been shown to be able to prevent and treat arthritis in studies:

1. Chondroitin: a major component of cartilage and a naturally occurring substance. Chondroitin can inhibit enzymes that degrade cartilage and therefore prevent degeneration of joints. It is synthesized by polymerization of many glucosamine monomers. Chondroitin can be absorbed directly by human body. Many clinical trials suggested that chondroitin can improve and treat osteoarthritis and is safe to use (some results see: Brief, A. A.; Maurer, S. G.; Di Cesare, P. E. J. Am. Acad. Orthop. Surg. 2001, 9, 71-78).

2. Hyaluronic acid: a naturally occurring major component of synovial fluid. Its physiological functions include lubrication of joint and absorption of vibration. Hyaluronic acid is a polysaccharide consisting of repeated N-acetyl-D-glucosamine and D-glucuronic acid units. Its molecular weights vary from 160-240 kDa, 500-800 kDa and greater than 800 kDa, which are called low, median, and high molecular weight hyaluronic acids, respectively. Generally, hyaluronic acids with higher molecular weights show better activities. It was clinically proven that hyaluronic acid sodium salts can relieve pain and improve joint function of osteoarthritis patients when injected into the joints (Altman, R.; Moskowitz, R. J Rheumatol. 1998, 25, 11 and Huskisson, E. C.; Donnelly, S. J. Rheumatol. 1999, 38, 602-607).

3. Methyl sulfonylmethane (MSM): an organosulfur compound. It occurs naturally in meat, seafood, vegetable, fruit, coffee, tea, etc. Studies showed that joints of osteoarthritis have lower level of sulfur than normal ones. On the other hand, sulfur is a necessary element for synthesizing cartilage and synovial fluid. MSM is a source of bio-absorbable sulfur. It can help relieve pain and inflammation of arthritis patients and prevent degradation of cartilage.

4. Diacerein: diacetyl derivative of naturally occurring rhein. It has anti-inflammatory activities and can protect the cartilage through inhibiting interleukin-1β and metalloproteinases and controlling production and release of oxygen free radicals. It was proven that diacerein can significantly alleviate the symptoms of osteoarthritis patients in a 3-year-long clinical trial. The adverse effect that diacerein showed is only a short-term diarrhea. Diacerein has been classified as slow-acting drug for osteoarthritis.

5. Vitamins A, C, D, and E: Vitamins A, C, and E are three major antioxidants in foods. These vitamins have been shown clinically to slow down the progression of osteoarthritis through their anti-oxidative properties. Vitamin D helps prevent osteoarthritis through calcification and bone and cell division.

Osteoarthritis has a close relationship with osteoporosis, which is caused by deficiency or loss of calcium in bones. No joint is healthy if its attached bone is unhealthy. In other words, a person with healthy and strong bones has less chance to develop osteoarthritis. Calcium and phosphorus are two fundamental elements, which forms bones. Calcium orotate can easily cross cell membrane and is therefore a highly absorbable calcium compound. About 30 years ago, calcium orotate was first found to be very effective in treating Tietze syndrome due to its special affinity with cartilage. Cartilage is also the place where calcium orotate gets metabolized. Therefore this compound is a good source of calcium for cartilage (Nieper, H. A. Z. Prakl. Geriatrie 1973, 3(4), 82-89). Calcium orotate has almost no known side effect. Other absorbable calcium sources include calcium lactate, calcium citrate, calcium ascorbate, calcium salts of amino acids etc or calcium carbonate plus corresponding organic acids. Phosphoric acid and calcium phosphate are sources of phosphorus. The best effect to maintain a healthy bone can be achieved only when calcium and phosphorus are supplemented at the same time.

Currently, almost all marketed glucosamine products are in forms of capsule, pill or tablet. Not only is the big capsule or tablet hard to swallow, but the worst thing is that solid form glucosamine is difficult to be absorbed by human body. It is pointed out in Physician's Desk Reference (50^{th} ed., pp. 1542, 1996; Publisher: Medical Economics Company, Montvale, N.J.) that “only 10-20% of vitamins and minerals in any pill form is absorbed by the human body.” Furthermore, the National Advisory Board states that 100 mg consumed in tablet form translates to a minute stabilized 8.3 mg or 8.3% concentrated in the blood, as compared to 98% in liquid form.

### CONTENTS OF THE INVENTION

The purpose of this invention is to overcome the low absorption of solid form glucosamine products that are already on the market and to provide a series of easily absorbable joints-protecting beverages and/or foods.

In order to achieve this goal, the methods used are described herein as: Add one or more of the following cartilage- and synovial fluid-protecting and bone-enhancing active ingredients to commercially available beverages and/or foods: glucosamine sulfate or hydrochloride; chondroitin sulfate; methyl sulfonylmethane; hyaluronic acid; diacerein; vitamin A; vitamin C; vitamin D; vitamin E; calcium orotate; calcium lactate; calcium ascorbate or calcium carbonate plus ascorbic acid; calcium citrate or calcium carbonate plus citric acid; calcium phosphate or calcium carbonate plus phosphoric acid; or salts, derivatives, or metabolites of the above-mentioned substances, which have similar biological activities, such as metal ion containing salts of N-acetyl glucosamine, glucosamine, chondroitin, hyaluronic acid, etc.

Suitable amounts of active ingredients for one person to consume in one day are as follows:

| | | |
|---|---|---|
| Glucosamine sulfate or hydrochloride or combinations | 1-5000 | mg; |
| of these two: | | |
| Chondroitin sulfate: | 0-4000 | mg; |
| Methyl sulfonylmethane: | 0-1500 | mg; |
| Hyaluronic acid: | 0-4800 | mg; |
| Diacerein: | 0-500 | mg; |
| Vitamin A or C or D or E or their | 0-2500 | mg; |
| combinations: | | |
| Calcium orotate: | 0-4800 | mg; |
| Calcium lactate: | 0-4800 | mg; |
| Calcium ascorbate or calcium carbonate | 0-4800 | mg; |
| plus ascorbic acid: | | |
| Calcium citrate or calcium carbonate | 0-4800 | mg; |
| plus citric acid: | | |
| Calcium phosphate or calcium carbonate | 0-3500 | mg. |
| plus phosphoric acid: | | |

The above active ingredients also include their salts, derivatives, and metabolites with similar activities. For example, metal ion containing salts of N-acetyl glucosamine, glucosamine, chondroitin, hyaluronic acid, etc.

The beverages and/or foods described in this invention can be in the following forms: liquid, liquid-solid suspension, gelatin, semi-solid, or solid. Among them, liquids, solid-liquid suspensions and semi-solids include but not limited to: carbonated or non-carbonated, concentrated or instantly drinkable waters (such as distilled water, mineral water, boiled water, etc); water-based drinks (such as cola, soda, carbonated water, etc); fruit juices (such as coconut juice, apple juice, grape juice, orange juice, banana juice, mango juice, peach juice, plum juice, litchi juice, kiwi juice, pear juice, tangerine juice, pineapple juice, sugarcane juice, apricot juice, strawberry juice, or other fruit juice, or combinations of two or more of these juices); vegetable juices (such as tomato juice, cabbage juice, green vegetable juice, or other vegetable juices, or combinations of two or more of these juices); animal milks (such as cow milk, goat milk, etc) and milk products (such as yogurt, cheese, ice cream, cake, etc); teas and tea products (such as liquid green tea, red tea, black tea, oolong tea, jasmine tea, etc or mixtures of two or more of these teas); alcohol-containing liquids (such as liquor, beer, wine, yellow wine, red wine, rice wine, fermented sweet rice, etc or mixtures of two or more of them); honeys (such as honeybee honey, etc); beans and bean products (such as soybean milk, soybean-based drink, soybean drop, bean curd, fermented bean curd, etc); jams (such as strawberry jam, peanut jam, etc); mixed congees, etc. Among them, solids and gelatins include but not limited to: ice-based drinks (such as ice, ice cream, flavored ice, etc); sugars and sugar products (such as white sugar, crystal sugar, brown sugar, etc or their mixtures. These sugars can be in the following forms: soft sugar, hard sugar, milk sugar, rubber sugar, cotton sugar, gelatin sugar, sticky sugar, etc); milk powders (such as whole milk powder, skim milk powder, baby milk powder etc); solid drinks (such as Mai Ru Jing (extract of malt and milk), Guo Zheng (concentrated fruit powder), coffee, cocoa, ginseng powder/extract, etc or combinations of one or more of these substances); teas and tea products (such as green tea, red tea, black tea, oolong tea, jasmine tea, etc or combinations of two or more of these teas or a mixture of tea, the fruit of Chinese wolfberry, dates, etc to form so called San Pao Tai); chocolates; oatmeals; cereals; sesame products (i.e., so-called Zhi Ma Hu); soybean products; corn products; oats products; rice products; wheat products; starch products, etc.

The beverages and/or foods described in this invention are prepared by using either of the following two methods. The first method is as follows: Add one or more of the following active ingredients according to recommended amounts to commercially available beverages and/or foods to make joint-protecting beverages and/or foods: glucosamine sulfate or hydrochloride; chondroitin sulfate; methyl sulfonylmethane; hyaluronic acid; diacerein; vitamin A; vitamin C; vitamin D; vitamin E; calcium and phosphorus additives. Glucosamine sulfate or hydrochloride or combinations of these two is mandatory ingredient. Chondroitin sulfate, methyl sulfonylmethane, and hyaluronic acid are the 2^{nd}, 3^{rd}, and 4^{th} priority ingredients, respectively. Addition of other active ingredients is optional. The active ingredients are added into commercial beverages and/or foods as either solids or aqueous solutions. After addition, the mixture is stirred to make everything dissolved and homogenous. This process is usually carried out at ambient temperature (0° C.-37° C.). Then the pH values and taste of the above mixture are tested. If necessary, one or more of the following substances can be optionally added to achieve the best taste and nutrition: sweeteners; pigments; flavoring essences; preservatives; vitamins; minerals; rare elements; proteins; amino acids; fats; carbohydrates etc. The next step is to control the growth of microorganisms. Either heat pasteurization or radiation sterilization are used, more preferably the former. When heat pasteurization process is used, the temperatures are kept between 50° C.-90° C. and time is 10 controlled between 1 minute to 30 minutes, more preferably 70° C.-80° C. for 10 seconds to 3 minutes. The last step is bottling, which is usually carried out at ambient temperature. When radiation sterilization is used, the process is usually performed at ambient temperature. Carbonation of the final products is optional.

The second method for preparing joint-protecting beverages and/or foods is as follows: Mix active ingredients according to recommended amounts with non-active ingredients together. Active ingredients are one or more substances selected from glucosamine sulfate or hydrochloride; chondroitin sulfate; methyl sulfonylmethane; hyaluronic acid; diacerein; vitamin A; vitamin C; vitamin D; vitamin E; calcium and phosphorus additives. Liquid or solid-liquid suspension beverages are water-based with weight percentage of water greater than 70%. Solid beverages are mixtures of one or more of above-mentioned active ingredients with non-active ingredients. Glucosamine sulfate or hydrochloride or combinations of these two is mandatory ingredient. Chondroitin sulfate, methyl sulfonylmethane, and hyaluronic acid are the 2^{nd}, 3^{rd}, and 4^{th} priority ingredients, respectively. Addition of other active ingredients is optional. The final products have pH values ranged between 2-10, more preferably neutral to acidic, i.e. pH=2-7. In order to achieve this, food industry-acceptable organic acids, such as citric acid, lactic acid, etc, and inorganic bases, such as sodium bicarbonate, sodium carbonate, sodium hydroxide, etc, are used to adjust the pH value of products. If necessary, preservatives such as sodium benzoate can be used for preserving purpose; natural or artificial pigments can be used to adjust the color of products; flavoring essences can be used to adjust the taste of products; white sugar, brown sugar, or crystal sugar can be used as calorie sources; sweeteners such as saccharin can be used to make low-calorie beverages and/or foods. Addition of vitamins and minerals is optional. To control the growth of microorganisms, either heat pasteurization or radiation sterilization are used, more preferably the former. When heat pasteurization process is used, the temperatures are kept between 50° C.-90° C. and time is controlled between 1 minute to 30 minutes, more preferably 70° C.-80° C. for 10 seconds to 3 minutes. Bottling is usually carried out at ambient temperature. When radiation sterilization is used, the process is usually performed at ambient temperature. Carbonation of the final products is optional. Final products can be instantly drinkable or concentrated (It can be diluted with water by consumer before drinking); normal-calorie or diet (low-calorie); in liquid form or suspension or solid. The fruit of Chinese wolfberry, dates, ginseng, etc can be used in some solid beverages.

Beverages and/or foods described in this invention have both preventing and treating effects for osteoarthritis and are suitable for long-term consumption. Beverages and/or foods described in this invention are taken in liquid form and are therefore absorbed and utilized thoroughly by human body. Beverages and/or foods described in this invention not only can relieve pain and alleviate other clinical symptoms of osteoarthritis patients, but can also repair damaged joints, slow down or prevent degeneration of cartilage and synovial fluid, and therefore act directly on the real cause of osteoarthritis, not like NSAIDs drugs, which can only improve symptoms. For normal healthy people especially who is 30 or older, consuming the beverages and/or foods described in this invention can prevent degeneration of his/her joints and therefore lower the chance to develop osteoarthritis. The ability for human body to generate glucosamine decreases with age increases. If glucosamine cannot be supplemented externally when one becomes older, his/her joints begin to degenerate due to lacking of nutrition, and eventually develop osteoarthritis. The foods one eats daily don't contain glucosamine. So it is not possible for preventing degeneration of one's cartilage and synovial fluid by just eating regular foods. If a person with healthy joints consumes the beverages and/or foods described in this invention, the chance for him/her to develop osteoarthritis becomes much lower because his/her joints are always supplied with enough amount of necessary nutrition.

The beverages and/or foods described in this invention are able to repair damaged joints caused by overuse in athletic training or heavy physical labor and restore lost functions. Certain groups of people, such as athletes, dockers, miners, farmers, etc, are routinely working under high intensity of labor. So the chance for their joints to get overused and injured is high. The beverages and/or foods described in this invention can provide their joints with necessary nutrition and protection.

In addition to human beings, the beverages and/or foods described in this invention are also suitable for other mammals, such as dog, cat, cow/ox, sheep/goat, pig, rat/mouse, squirrel, etc, to consume. The physiological functions of the beverages and/or foods described in this invention in mammals are similar to those in human beings.

In order to help people further understand this invention, the following examples are provided. However, in no way should these examples be used to limit the contents or claims of this invention. The compositions and the amounts and numbers of either active or non-active ingredients shown in these examples can be changed in order that best taste and nutrition are achieved.

### EXAMPLES 1-18

Preparations of novel cartilage-, synovial fluid-, and bone-enhancing beverages and/or foods for preventing or treating osteoarthritis by adding one or more of active ingredients selected from glucosamine sulfate or hydrochloride; chondroitin sulfate; methyl sulfonylmethane; hyaluronic acid; and calcium and phosphorus additives to commercially available liquid beverages or foods:

To a container equipped with stirrer was placed 400 mL of commercially available beverages or foods. Glucosamine sulfate; glucosamine hydrochloride; chondroitin sulfate; methyl sulfonylmethane; hyaluronic acid; and calcium orotate were then added to this container according to amounts shown in Table 1. The resulting mixture was stirred to make active ingredients dissolved. Filtration of the mixture is optional. After pastured or sterilized, the mixture was cooled, carbonated or non-carbonated, and bottled to afford about 390-415 mL final products. Examples 1-3 need to be carbonated. Filtration is needed in Examples 1-3 and 13-15. Recommended amounts for an adult to consume in one day are: 400 mL for Examples 4, 11 and 14; 100 mL for Examples 15-17; 1200 mL for all other Examples.

TABLE 1
Preparation of Liquid or
Liquid-solid Suspension Drinks and Foods:

| | | Active ingredients and their amounts | | | | | |
|---|---|---|---|---|---|---|---|
| | | added to 400 mL of commercial drink (in mg) | | | | | |
| | Commercial | | | | | Methyl | |
| Exam- | drink | Glucosa- | Glucosa- | Chondroi- | Hyaluro- | Sulfonyl- | Calcium |
| ple | (400 mL) | mine HCl | mine Sulfate | tin Sulfate | nic acid | methane | Orotate |
| 1 | Coca-Cola | 208 | 208 | 0 | 0 | 0 | 200 |
| 2 | Pepsi Cola | 208 | 208 | 0 | 0 | 0 | 200 |
| 3 | Jianlibao Cola | 208 | 208 | 0 | 0 | 0 | 200 |
| 4 | Milk | 1250 | 0 | 1000 | 1000 | 416 | 0 |
| 5 | Coconut juice | 208 | 208 | 0 | 0 | 0 | 200 |
| 6 | Soybean milk | 625 | 0 | 500 | 500 | 208 | 150 |
| 7 | Orange juice | 208 | 208 | 166 | 166 | 70 | 200 |
| 8 | Apple juice | 208 | 208 | 166 | 166 | 70 | 200 |
| 9 | Grape juice | 208 | 208 | 166 | 166 | 70 | 200 |
| 10 | Mango juice | 208 | 208 | 166 | 166 | 70 | 200 |
| 11 | Sugarcane | 516 | 516 | 332 | 332 | 70 | 400 |
| | juice | | | | | | |
| 12 | Tomato juice | 208 | 208 | 166 | 166 | 70 | 200 |
| 13 | Liquid green | 208 | 208 | 0 | 166 | 0 | 0 |
| | tea | | | | | | |
| 14 | Beer | 516 | 516 | 0 | 0 | 0 | 0 |
| 15 | Wine | 2500 | 2500 | 0 | 0 | 0 | 0 |
| 16 | Honey | 2500 | 2500 | 0 | 0 | 0 | 1000 |
| 17 | Strawberry | 2500 | 2500 | 0 | 0 | 0 | 1000 |
| | jam | | | | | | |
| 18 | Mixed congee | 208 | 208 | 200 | 200 | 0 | 100 |

### Example 19

Same as Example 3 except that 30 mg of diacerein and 300 mg of vitamins A, C, D, and E (1:1:1:1 ratio in weight) were added.

### EXAMPLE 20

Same as Example 8 except that 30 mg of diacerein was added.

### EXAMPLES 21-28

Preparations of novel cartilage-, synovial fluid-, and bone-enhancing beverages or foods for preventing or treating osteoarthritis by adding one or more active ingredients selected from glucosamine sulfate or hydrochloride; chondroitin sulfate; methyl sulfonylmethane; hyaluronic acid; and calcium and phosphorus additives to commercially available solid drinks: To a container equipped with stirrer was placed 100 g of commercially available solid beverages and/or foods. Glucosamine sulfate; glucosamine hydrochloride; chondroitin sulfate; methyl sulfonylmethane; hyaluronic acid; and calcium orotate were then added to this container according to amounts shown in Table 2. The resulting mixture was stirred to make everything mixed well. Smashing or sieving is needed in some cases. Sterilization and packaging lead to final products. Sieving is needed for Examples 21-24, 26, and 28. Recommended amounts for an adult to consume in one day are: 100 g for Examples 21-23; 50 mL for Examples 24-26; 200 g for Examples 27-28.

TABLE 2
Preparation of Solid Drinks and Foods:

| | | Active ingredients and their amounts | | | | | |
|---|---|---|---|---|---|---|---|
| | | added to 1400 g of commercial drink or food (in mg) | | | | | |
| | Commercial | | | | | Methyl | |
| Exam- | drink | Glucosa- | Glucosa- | Chondroi- | Hyaluro- | Sulfonyl- | Calcium |
| ple | (100 g) | mine HCl | mine Sulfate | tin Sulfate | nic acid | methane | Orotate |
| 21 | White sugar | 500 | 500 | 800 | 0 | 0 | 500 |
| 22 | Brown sugar | 500 | 500 | 800 | 0 | 0 | 500 |
| 23 | Mai Ru Jing | 500 | 500 | 800 | 800 | 0 | 500 |
| | (i.e., extract | | | | | | |
| | of malt and | | | | | | |
| | milk | | | | | | |
| 24 | Fruit juice | 1000 | 1000 | 1600 | 800 | 0 | 1000 |
| | powder | | | | | | |
| 25 | Green tea* | 1000 | 1000 | 1600 | 0 | 0 | 1000 |
| 26 | Coffee | 1000 | 1000 | 1600 | 0 | 0 | 1000 |
| 27 | Oatmeal | 250 | 250 | 400 | 400 | 0 | 100 |
| 28 | Zhi Ma Hu | 250 | 250 | 400 | 400 | 0 | 100 |
| | (Sesame | | | | | | |
| | product) | | | | | | |

*Adding additionally 30 g Fruit of Chinese Wolfberry, 100 g dates, and 100 g crystal sugar.

### EXAMPLE 29

Preparation of Active Ingredient Mixture I: To a container equipped with stirrer was placed 100 g of glucosamine sulfate, 100 g of glucosamine hydrochloride, 160 g of chondroitin sulfate, 67 g of methyl sulfonylmethane, 160 g of hyaluronic acid, and 180 g of calcium orotate. The mixture was stirred well and used directly in next step.

### EXAMPLE 30

Preparation of Active Ingredient Mixture II: To a container equipped with stirrer was placed 100 g of glucosamine sulfate, 100 g of glucosamine hydrochloride, 160 g of chondroitin sulfate, 67 g of methyl sulfonylmethane, and 160 g of hyaluronic acid. The mixture was stirred well and used directly in next step.

### EXAMPLE 31

Preparation of Active Ingredient Mixture III: To a container equipped with stirrer was placed 100 g of glucosamine sulfate, 100 g of glucosamine hydrochloride, 160 g of chondroitin sulfate, and 67 g of methyl sulfonylmethane. The mixture was stirred well and used directly in next step.

### EXAMPLE 32

Preparation of Drink Concentrate I: To 60 g of water was added sequentially high fructose corn syrup (200 g), citric acid (10 g), ascorbic acid (i.e., Vitamin C, 10 g), Arabic gel (5 g), sodium benzoate (1 g), and potassium sorbate (1 g). The mixture was stirred well and used directly in next step.

### EXAMPLE 33

Preparation of Drink Concentrate III: To 60 g of water was added sequentially high fructose corn syrup (200 g), citric acid (10 g), ascorbic acid (i.e., Vitamin C, 10 g), sodium benzoate (1 g), potassium sorbate (1 g) and orange juice concentrate (40 g). The mixture was stirred well and used directly in next step.

### EXAMPLE 34

Preparation of Drink Concentrate III: To 60 g of water was added sequentially high fructose corn syrup (200 g), citric acid (10 g), ascorbic acid (i.e., Vitamin C, 10 g), sodium benzoate (1 g), potassium sorbate (1 g), and apple juice concentrate (40 g). The mixture was stirred well and used directly in next step.

### EXAMPLE 35

Preparation of Drink Concentrate IV: To 60 g of water was added sequentially high fructose corn syrup (200 g), citric acid (10 g), ascorbic acid (i.e., Vitamin C, 10 g), sodium benzoate (1 g), potassium sorbate (1 g), and grape juice concentrate (40 g). The mixture was stirred well and used directly in next step.

### EXAMPLE 36

Preparation of Drink Concentrate V: To 60 g of water was added sequentially high fructose corn syrup (200 g), citric acid (10 g), ascorbic acid (i.e., Vitamin C, 10 g), sodium benzoate (1 g), potassium sorbate (1 g), and squeezed tomato juice (100 g). The mixture was stirred well and used directly in next step.

### EXAMPLE 37

Preparation of Drink Concentrate VI (low-calorie type): To 200 g of water was added sequentially white sugar (30 g), saccharin (0.3 g), citric acid (10 g), ascorbic acid (i.e., Vitamin C, 10 g), Arabic gel (5 g), sodium benzoate (1 g) and potassium sorbate (1 g). The mixture was stirred well and used directly in next step.

### EXAMPLE 38

Preparation of joint-protecting sodas: To a container equipped with stirrer was added 60 g of Drink Concentrate I (Example 32) and 1.6 g of Active Ingredient Mixture I or II or III (Example 29, 30, or 31). The resulting mixture was diluted with water to 400 mL. Necessary amounts of pigment and flavoring essence were optionally added to adjust the taste to different styles, such as orange, grape taste, etc. Necessary amount of white sugar was used to adjust the degree of sweetness. The pH was adjusted with citric acid to 2.2-3.0. The resulting solution was stirred and quickly heated to 80° C. and kept for 1 minute, then rapidly cooled to ambient temperature. Carbonation and bottling gave the final product.

### EXAMPLE 39

Preparation of joint-protecting fruit-flavoring drinks: To a container equipped with stirrer was added 60 g of Drink Concentrate II or III or IV or V (Examples 33-36) and 1.6 g of Active Ingredient Mixture I or II or III (Example 29, 30, or 31). The resulting mixture was diluted with water to 400 mL. Necessary amounts of pigment and flavoring essence were added to adjust the taste to different styles, such as orange, apple, grape taste, etc. If necessary, white sugar was used to adjust the degree of sweetness. The pH was adjusted with citric acid to 2.2-3.0. The resulting solution was stirred and quickly heated to 80° C. and kept for 1 minute, then rapidly cooled to ambient temperature. Bottling gave the final product.

### EXAMPLE 40

Preparation of joint-protecting diet drinks: To a container equipped with stirrer was added 60 g of Drink Concentrate VI (Example 37) and 1.6 g of Active Ingredient Mixture I or II or III (Example 29, 30, or 31). The resulting mixture was diluted with water to 400 mL. Necessary amounts of pigment and flavoring essence were optionally added to adjust the taste to different styles, such as orange, apple, grape taste, etc. If necessary, saccharin was used to adjust the degree of sweetness. The pH was adjusted with citric acid to 2.2-3.0. The resulting solution was stirred and quickly heated to 80° C. and kept for 1 minute, then rapidly cooled to ambient temperature. Carbonation and bottling gave the final product.

What is claimed is:

1. A joint-protecting beverage and/or food comprising one or more of active ingredients selected from:
glucosamine sulfate or glucosamine hydrochloride; chondroitin sulfate; methyl sulfonylmethane (MSM); hyaluronic acid; diacerein; vitamin A; vitamin C; vitamin D; vitamin E; calcium orotate; calcium lactate; calcium ascorbate or calcium carbonate plus ascorbic acid; calcium citrate or calcium carbonate plus citric acid; calcium phosphate or calcium carbonate plus phosphoric acid; or salts, derivatives, or metabolites of the above-mentioned substances, which have similar biological activities.

2. The beverage and/or food according to claim 1, wherein the suitable amounts of said active ingredients for a person to consume in one day are as follows:

| | | |
|---|---|---|
| glucosamine sulfate or hydrochloride or | 1-5000 | mg; |
| combinations of these two: | | |
| chondroitin sulfate: | 0-4000 | mg; |
| methyl sulfonylmethane: | 0-1500 | mg; |
| hyaluronic acid: | 0-4800 | mg; |
| diacerein: | 0-500 | mg; |
| vitamin A or C or D or E or their | 0-2500 | mg; |
| combinations: | | |
| calcium orotate: | 0-4800 | mg; |
| calcium lactate: | 0-4800 | mg; |
| calcium ascorbate or calcium carbonate | 0-4800 | mg; |
| plus ascorbic acid: | | |
| calcium citrate or calcium carbonate plus | 0-4800 | mg; |
| citric acid: | | |
| calcium phosphate or calcium carbonate | 0-3500 | mg. |
| plus phosphoric acid: | | |

3. The beverage and/or food according to claim 1 further comprising ingredients selected from proteins; amino acids; fats; carbohydrates; vitamins; minerals; rare elements;
sweeteners; pigments; flavoring essences; or combinations thereof.

4. The beverage and/or food according to claim 1 can be in the following forms: liquid; solid-liquid suspension; gelatin; semi-solid; or solid.

5. The beverage and/or food according to claim 4, wherein liquids, solid-liquid suspensions and semi-solids include but not limited to: waters; water-based drinks; fruit juices; vegetable juices; animal milks and milk products; teas and tea products; alcohol-containing liquids; honeys; beans and bean products; jams; mixed congees, etc. Solids and gelatins include but not limited to: ice-based drinks; sugars and sugar products; milk powders; solid drinks; teas and tea products or a mixture of tea, fruit of Chinese wolfberry and dates (i.e., so-called San Pao Tai); chocolates; oatmeals; cereals; sesame products (i.e., so-called Zhi Ma Hu); soybean products; corn products; oats products; rice products; wheat products; starch products.

6. The beverage and/or food according to claim 1 can be carbonated or non-carbonated;
can be ready-for-drinking or concentrated; their pH values are between 2 to 10, more preferably 2 to 7.

7. A method of preparing a joint-protecting beverage and/or food comprising the steps of
A. providing a commercially available beverage and/or food;
B. providing one or more of active ingredients selected from: glucosamine sulfate or glucosamine hydrochloride; chondroitin sulfate; methyl sulfonylmethane (MSM); hyaluronic acid; diacerein; vitamin A; vitamin C; vitamin D; vitamin E; calcium orotate; calcium lactate; calcium ascorbate or calcium carbonate plus ascorbic acid; calcium citrate or calcium carbonate plus citric acid; calcium phosphate or calcium carbonate plus phosphoric acid; or salts, derivatives, or metabolites of the above-mentioned substances, which have similar biological activities;
C. adding said active ingredients to said beverage and/or food. The mixture is then stirred, pasteurized or sterilized to give the final products.

8. A method of preparing a joint-protecting beverage and/or food comprising the steps of
A. providing one or more of the following active ingredients according to amount listed below:

| | | |
|---|---|---|
| glucosamine sulfate or hydrochloride or | 1-5000 | mg; |
| combinations of these two: | | |
| chondroitin sulfate: | 0-4000 | mg; |
| methyl sulfonylmethane: | 0-1500 | mg; |
| hyaluronic acid: | 0-4800 | mg; |
| diacerein: | 0-500 | mg; |
| vitamin A or C or D or E or their | 0-2500 | mg; |
| combinations: | | |
| calcium orotate: | 0-4800 | mg; |
| calcium lactate: | 0-4800 | mg; |
| calcium ascorbate or calcium carbonate | 0-4800 | mg; |
| plus ascorbic acid: | | |
| calcium citrate or calcium carbonate | 0-4800 | mg; |
| plus citric acid: | | |
| calcium phosphate or calcium carbonate | 0-3500 | mg; |
| plus phosphoric acid: | | |

B. providing non-active ingredients;
C. mixing said active ingredients and said non-active ingredients to afford the final product.

9. A use of the beverage and/or food according to claim 1 is to prevent and treat osteoarthritis.

10. The use according to claim 9 is not only for human beings, but also suitable for other mammals.

11. The beverage and/or food according to claim 2 further comprising ingredients selected from proteins; amino acids; fats; carbohydrates; vitamins; minerals; rare elements; sweeteners; pigments; flavoring essences; or combinations thereof.

12. The beverage and/or food according to claim 2 can be in the following forms: liquid; solid-liquid suspension; gelatin; semi-solid; or solid.

13. The beverage and/or food according to claim 3 can be in the following forms: liquid; solid-liquid suspension; gelatin; semi-solid; or solid.

14. The beverage and/or food according to claim 11 can be in the following forms: liquid; solid-liquid suspension; gelatin; semi-solid; or solid.

15. The beverage and/or food according to claim 12, wherein liquids, solid-liquid suspensions and semi-solids include but not limited to: waters; water-based drinks; fruit juices; vegetable juices; animal milks and milk products; teas and tea products; alcohol-containing liquids; honeys; beans and bean products; jams; mixed congees, etc. Solids and gelatins include but not limited to: ice-based drinks; sugars and sugar products; milk powders; solid drinks; teas and tea products or a mixture of tea, fruit of Chinese wolfberry and dates (i.e., so-called San Pao Tai); chocolates; oatmeals; cereals; sesame products (i.e., so-called Zhi Ma Hu); soybean products; corn products; oats products; rice products; wheat products; starch products.

16. The beverage and/or food according to claim 13, wherein liquids, solid-liquid suspensions and semi-solids include but not limited to: waters; water-based drinks; fruit juices; vegetable juices; animal milks and milk products; teas and tea products; alcohol-containing liquids; honeys; beans and bean products; jams; mixed congees, etc. Solids and gelatins include but not limited to: ice-based drinks; sugars and sugar products; milk powders; solid drinks; teas and tea products or a mixture of tea, fruit of Chinese wolfberry and dates (i.e., so-called San Pao Tai); chocolates; oatmeals; cereals; sesame products (i.e., so-called Zhi Ma Hu); soybean products; corn products; oats products; rice products; wheat products; starch products.

17. The beverage and/or food according to claim 14, wherein liquids, solid-liquid suspensions and semi-solids include but not limited to: waters; water-based drinks; fruit juices; vegetable juices; animal milks and milk products; teas and tea products; alcohol-containing liquids; honeys; beans and bean products; jams; mixed congees, etc. Solids and gelatins include but not limited to: ice-based drinks; sugars and sugar products; milk powders; solid drinks; teas and tea products or a mixture of tea, fruit of Chinese wolfberry and dates (i.e., so-called San Pao Tai); chocolates; oatmeals; cereals; sesame products (i.e., so-called Zhi Ma Hu); soybean products; corn products; oats products; rice products; wheat products; starch products.

18. The beverage and/or food according to claim 2 can be carbonated or non-carbonated; can be ready-for-drinking or concentrated; their pH values are between 2 to 10, more preferably 2 to 7.

19. The beverage and/or food according to claim 3 can be carbonated or non-carbonated; can be ready-for-drinking or concentrated; their pH values are between 2 to 10, more preferably 2 to 7.

20. The beverage and/or food according to claim 11 can be carbonated or non-carbonated; can be ready-for-drinking or concentrated; their pH values are between 2 to 10, more preferably 2 to 7.
